(19) United States
(12) Patent Application Publication
(10) Pub. No.: US 2022/0133196 A1
(43) Pub. Date: May 5, 2022

(54) MENTAL AND PHYSICAL STATE DISPLAY DEVICE, MENTAL AND PHYSICAL STATE DISPLAY SYSTEM, MENTAL AND PHYSICAL STATE DISPLAY METHOD, PROGRAM, AND STORAGE MEDIUM

(71) Applicant: Murata Manufacturing Co., Ltd., Nagaokakyo-shi (JP)

(72) Inventors: Takumi Ono, Nagaokakyo-shi (JP); Toru Yabe, Nagaokakyo-shi (JP)

(21) Appl. No.: 17/494,050

(22) Filed: Oct. 5, 2021

(30) Foreign Application Priority Data
Oct. 29, 2020 (JP) 2020-181671

(51) Int. Cl. A61B 5/16; A61B 5/00; G16H 10/60; G16H 20/70; G16H 20/90; G16H 50/30

(52) U.S. Cl. CPC A61B 5/165; A61B 5/742; G16H 10/60; A61B 2560/02; G16H 20/90; G16H 50/30; G16H 20/70

(57) ABSTRACT

A mental and physical state display device allows a user to intuitively grasp his/her mental and physical state substantially in agreement with his/her individual perception. The display device includes a display unit; and a display-processing unit that displays, on the display unit, state-indicating graphics in chronological order in a monthly, weekly or daily calendar format, with the state-indicating graphics indicating a mental and physical state computed based on a result of measurement of biometric information of a user, and representing levels of the mental and physical state in a distinguishable manner by a change in the respective state-indicating graphic. The display-processing unit displays at least one of the state-indicating graphics that corresponds to a criterion of the mental and physical state as a criterial state-indicating graphic that is distinguishable from a remainder of the state-indicating graphics, with the criterion being set on a user-to-user basis.

### CROSS REFERENCE TO RELATED APPLICATIONS

The present application claims priority to Japanese Patent Application No. 2020-181671, filed Oct. 29, 2020, the entire contents of which are incorporated herein by reference.

### TECHNICAL FIELD

The present disclosure relates to a mental and physical state display device, a mental and physical state display system, a mental and physical state display method, a program, and a storage medium.

### BACKGROUND

Japanese Unexamined Patent Application, Publication No. 2017-221338 (hereinafter “Patent Document 1” discloses a technique to display a state of the autonomic nervous system of a user based on the user's biometric information. More specifically, the technique disclosed in Patent Document 1 acquires an action index indicating an action of the autonomic nervous system and a balance index indicating balance, with the indices being computed based on a result of a measurement of the user's biometric information. The technique disclosed therein further determines a size of a state-indicating graphic corresponding to the measurement result, based on the action index; determines an outer shape of the state-indicating graphic such that a zigzag of the outer shape becomes wider as the balance indicated by the balance index worsens; generates the state-indicating graphic based on the size and the outer shape; and displays the generated state-indicating graphic. With this technique, the user can grasp a state of his/her autonomic nervous system, and can instinctively grasp his/her autonomic balance.

Meanwhile, a technique has been developed to evaluate a mental and physical state of a user, such as a degree of stress or a degree of fatigue (e.g., a degree of brain fatigue or a degree of muscle fatigue), based on the user's biometric information. However, since the same degree of stress and the same degree of fatigue are perceived individually differently by different users, it can be difficult for a user to intuitively grasp his/her mental and physical state substantially in agreement with his/her individual perception.

### SUMMARY OF THE INVENTION

Therefore, it is an object of the exemplary embodiments of the present disclosure to provide a mental and physical state display device, a mental and physical state display system, a mental and physical state display method, a program, and a storage medium that enable a user to intuitively grasp his/her mental and physical state substantially in agreement with his/her individual perception.

In accordance with an exemplary embodiment of the present disclosure, a mental and physical state display device includes a display unit; and a display-processing unit that displays, on the display unit, state-indicating graphics in chronological order in a monthly calendar format, a weekly calendar format, or a daily schedule format. Moreover, the state-indicating graphics indicate a mental and physical state computed based on a result of measurement of biometric information of a user, and represent levels of the mental and physical state in a distinguishable manner by a change in the respective state-indicating graphic. The display-processing unit displays at least one of the state-indicating graphics that corresponds to a criterion of the mental and physical state as a criterial state-indicating graphic that is distinguishable from a remainder of the state-indicating graphics, with the criterion being set on a user-to-user basis.

Moreover, a mental and physical state display system is provided according to an exemplary embodiment of the present disclosure that includes a sensor that measures biometric information of a user; a computation device that computes a mental and physical state of the user based on a result of measurement of the biometric information; the above-described mental and physical state display device that displays, in the calendar or schedule format, the state-indicating graphics indicating the computed mental and physical state and the criterial state-indicating graphic corresponding to the criterion of the mental and physical state set on a user-to-user basis; and a host device that communicates with the mental and physical state display device. The computation device is provided to the sensor, the mental and physical state display device, or the host device, or is provided outside the sensor, the mental and physical state display device, and the host device.

Moreover, a mental and physical state display method is provided according to an exemplary embodiment of the present disclosure that includes displaying state-indicating graphics in chronological order in a monthly calendar format, a weekly calendar format, or a daily schedule format, where the state-indicating graphics indicate a mental and physical state computed based on a result of measurement of biometric information of a user, and represent levels of the mental and physical state in a distinguishable manner by a change in the respective state-indicating graphic. The displaying includes displaying at least one of the state-indicating graphics that corresponds to a criterion of the mental and physical state as a criterial state-indicating graphic such that the criterial state-indicating graphic is distinguishable from a remainder of the state-indicating graphics, with the criterion being set on a user-to-user basis.

Yet further, a program is disclosed according to an exemplary embodiment of the present disclosure that is configured to cause a computer to perform a function of displaying state-indicating graphics in chronological order in a monthly calendar format, a weekly calendar format, or a daily schedule format. Moreover, the state-indicating graphics indicates a mental and physical state computed based on a result of measurement of biometric information of a user, and represents levels of the mental and physical state in a distinguishable manner by a change in the respective state-indicating graphic. The program further causes the computer to perform a function of displaying at least one of the state-indicating graphics that corresponds to a criterion of the mental and physical state as a criterial state-indicating graphic that is distinguishable from a remainder of the state-indicating graphics, with the criterion being set on a user-to-user basis.

Moreover, a computer-readable storage medium is disclosed according to an exemplary embodiment of the present disclosure that stores the program described above.

The exemplary embodiments of the present disclosure enable a user to intuitively grasp his/her mental and physical state substantially in agreement with his/her individual perception.

### BRIEF DESCRIPTION OF THE DRAWINGS

FIG. 1 is a diagram illustrating a mental and physical state display system according to an exemplary embodiment;

FIG. 2 is a diagram illustrating a mental and physical state display device according to the exemplary embodiment;

FIG. 3 is a flowchart illustrating a mental and physical state-displaying operation performed by the mental and physical state display system and the mental and physical state display device according to the exemplary embodiment;

FIG. 4A is a diagram illustrating an example of what is displayed by the mental and physical state display device of FIG. 2;

FIG. 4B is a diagram illustrating an example of what is displayed by the mental and physical state display device of FIG. 2;

FIG. 4C is a diagram illustrating an example of what is displayed by the mental and physical state display device of FIG. 2;

FIG. 5 is a diagram illustrating an example of what is displayed by the mental and physical state display device of FIG. 2;

FIG. 6A is a diagram illustrating an example of a state-indicating graphic;

FIG. 6B is a diagram illustrating an example of the state-indicating graphic;

FIG. 6C is a diagram illustrating an example of the state-indicating graphic;

FIG. 7A is a diagram illustrating an example of the state-indicating graphic;

FIG. 7B is a diagram illustrating an example of the state-indicating graphic;

FIG. 7C is a diagram illustrating an example of the state-indicating graphic;

FIG. 8 is a diagram illustrating an example of what is displayed by the mental and physical state display device of FIG. 2;

FIG. 9 is a diagram illustrating an example of what is displayed by the mental and physical state display device of FIG. 2;

FIG. 10 is a diagram illustrating an example of what is displayed by the mental and physical state display device of FIG. 2;

FIG. 11 is a diagram illustrating an example of what is displayed by the mental and physical state display device of FIG. 2; and

FIG. 12 is a diagram illustrating an example of what is displayed by the mental and physical state display device of FIG. 2.

### DETAILED DESCRIPTION OF THE EMBODIMENTS

An example of exemplary embodiments of the present disclosure will be described below with reference to the accompanying drawings. Note that in the drawings, the same or equivalent parts will be denoted by the same reference characters.

<Mental and Physical State Display System>

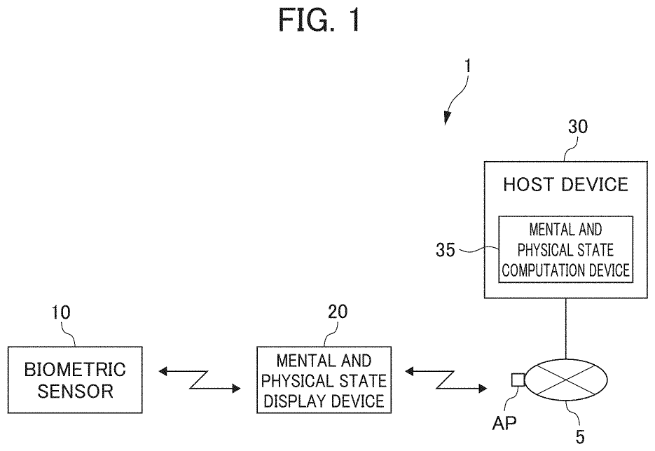

FIG. 1 is a diagram illustrating a mental and physical state display system according to the exemplary embodiment. The mental and physical state display system 1 illustrated in FIG. 1 includes a biometric sensor 10 (hereinafter, also referred to or abbreviated as the sensor), a mental and physical state display device 20 (hereinafter also referred to or abbreviated as the display device), a host device 30, and a mental and physical state computation device 35 (hereinafter also referred to or abbreviated as the computation device). The mental and physical state display system 1 displays, based on biometric information of a user, a mental and physical state of the user, such as a degree of stress and a degree of fatigue (e.g., a degree of brain fatigue, a degree of muscle fatigue, etc.), in the form of a graphic that is intuitive to understand.

Examples of the biometric sensor 10 include sensor devices, such as a heart rate meter, an electrocardiograph, a pulse rate meter, a pulse wave meter, a sphygmomanometer, a thermometer, and the like. The sensor 10 measures the biometric information of the user, under the control of the display device 20. The sensor 10 transmits the measured biometric information to the display device 20.

Examples of the mental and physical state display device 20 include information processing devices, such as a smartphone, a tablet, a PC, and the like. When the user measures his/her biometric information, the display device 20 controls measurement of the user's biometric information by the sensor 10, and receives a result of measurement of the user's biometric information from the sensor 10. The display device 20 transmits the result of measurement of the user's biometric information to the host device 30 through a network 5, and receives a mental and physical state computed based on the result of measurement of the user's biometric information, from the host device 30. Details of the display device 20 will be described later.

Examples of the host device 30 include information processing devices, such as a tablet and a PC. The host device 30 receives the result of measurement of the user's biometric information from the display device 20 through the network 5, and transmits the mental and physical state computed based on the result of measurement of the user's biometric information to the display device 20.

The mental and physical state computation device 35 computes the mental and physical state based on the result of measurement of the user's biometric information. For example, the computation device 35 computes a state of the user's autonomic nervous system based on the user's biometric information, such as heartbeat and a pulse, computes a degree of brain fatigue and/or a degree of physical fatigue based on an intensity of an action of the user's autonomic nervous system, and computes a degree of stress based on balance between the user's sympathetic nervous system and parasympathetic nervous system. Here, “a degree of stress” refers to a stress level that the user feels due to stress given from an external environment, for example. Examples of the computation device 35 include a known fatigue-stress measurement device.

The sensor 10 and the display device 20 can be configured to comply with any communication standard, examples of which include the so-called near-field wireless communication standards, such as Bluetooth® and Wi-Fi®. The display device 20 and the host device 30 can be configured to comply with any communication standard, examples of which include wireless communication standards, such as a wireless local area network (wireless LAN), long term evolution (LTE), and 3G that are implemented through an access point AP and the network 5; and wired communication standards, such as wired LAN.

In the present embodiment, an example in which the computation device 35 is provided to the host device 30 is illustrated. However, the mental and physical state computation device 35 may be provided to the display device 20 or the sensor 10. Alternatively, the computation device 35 may be provided outside the sensor 10, the display device 20, and the host device 30.

In the present embodiment, an example in which the sensor 10 is provided outside the display device 20 is illustrated. However, for example, in a case where the display device 20 is configured as a wearable terminal, the sensor 10 may be provided to the display device 20.

<<Mental and Physical State Display Device>>

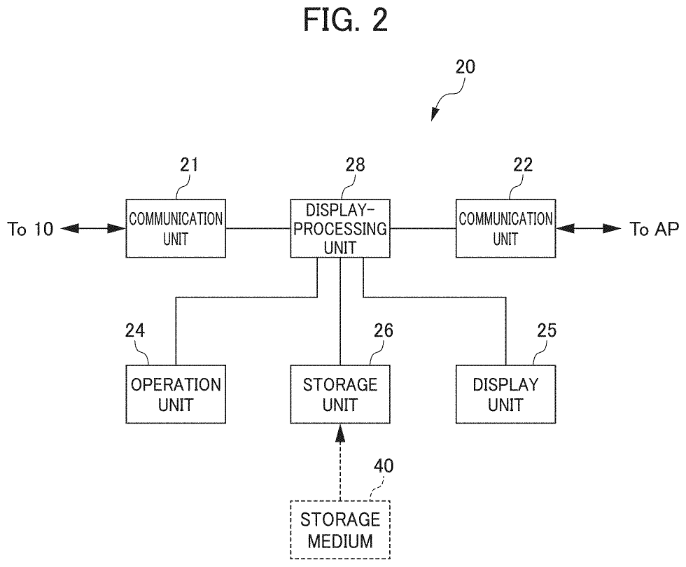

In the following, the mental and physical state display device 20 will be described in detail. FIG. 2 is a block diagram illustrating the mental and physical state display device 20 according to the present embodiment. As shown, the mental and physical state display device 20 illustrated in FIG. 2 includes a communication unit 21, a communication unit 22, an operation unit 24, a display unit 25, a storage unit 26, and a display-processing unit 28.

In the exemplary aspect, the communication unit 21 is configured to wirelessly communicate with the sensor 10. The communication unit 21 is an interface that complies with the so-called near-field communication standard, such as Bluetooth® or Wi-Fi®. It is noted that the communication standard with which the communication unit 21 complies is not limited to this.

The communication unit 22 is configured to communicate with the host device 30 through, for example, the access point AP and the network 5. In this aspect, the communication unit 22 is an interface that performs wireless communication in accordance with a communication standard such as wireless LAN, LTE, or 3G. It is noted that the communication unit 21 is not limited to this interface, but may be an interface that performs wired communication according to a communication standard such as wired LAN.

The operation unit 24 is a unit operable by a user. The operation unit 24 includes, for example, a keyboard having physical operation buttons, a mouse, or a touch panel having virtual operation buttons.

Moreover, the display unit 25 is configured to display information to the user, under the control of the display-processing unit 28. The display unit 25 includes, for example, a liquid crystal display or an organic EL display.

The storage unit 26 is configured to store, for example, schedule information and memo information (e.g., action information) that are set by the user. The storage unit 26 further stores, for example, preset inquiry information and preset recommendation information. The memo information (e.g., action information), the inquiry information, and the recommendation information will be described in detail later.

The storage unit 26 further stores programs (e.g., applications) that are executed by the display-processing unit 28 or data. The storage unit 26 includes a recording medium, such as a read only memory (ROM), a hard disk drive (HDD), a solid state drive (SSD), or a removable memory card.

The display-processing unit 28 displays, on the display unit 25, state-indicating graphics that indicate a mental and physical state of the user, the mental and physical state being computed based on the result of measurement of the user's biometric information. The mental and physical state includes at least one of a degree of stress or a degree of fatigue. The degree of fatigue includes a degree of brain fatigue and a degree of muscle fatigue.

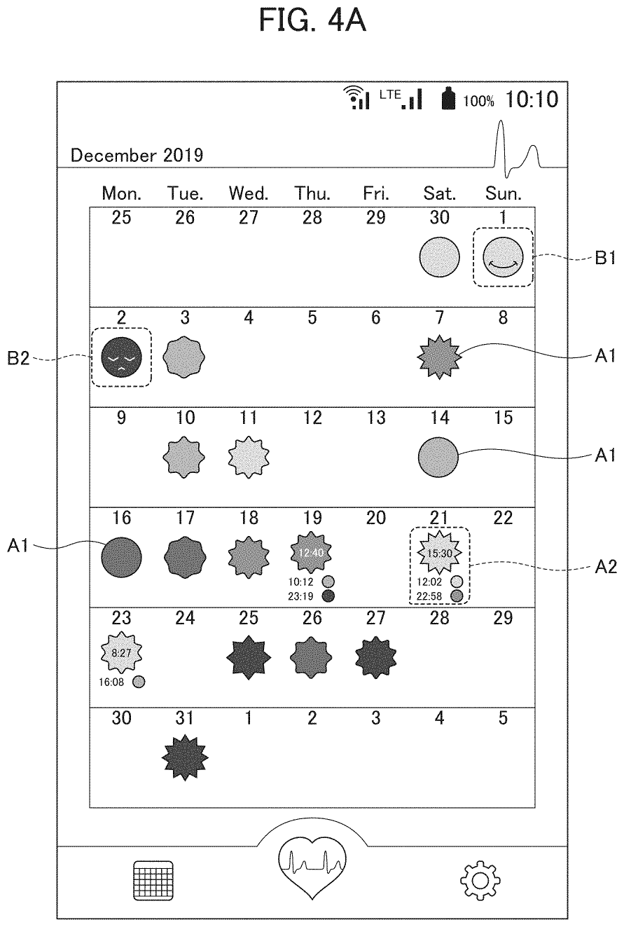

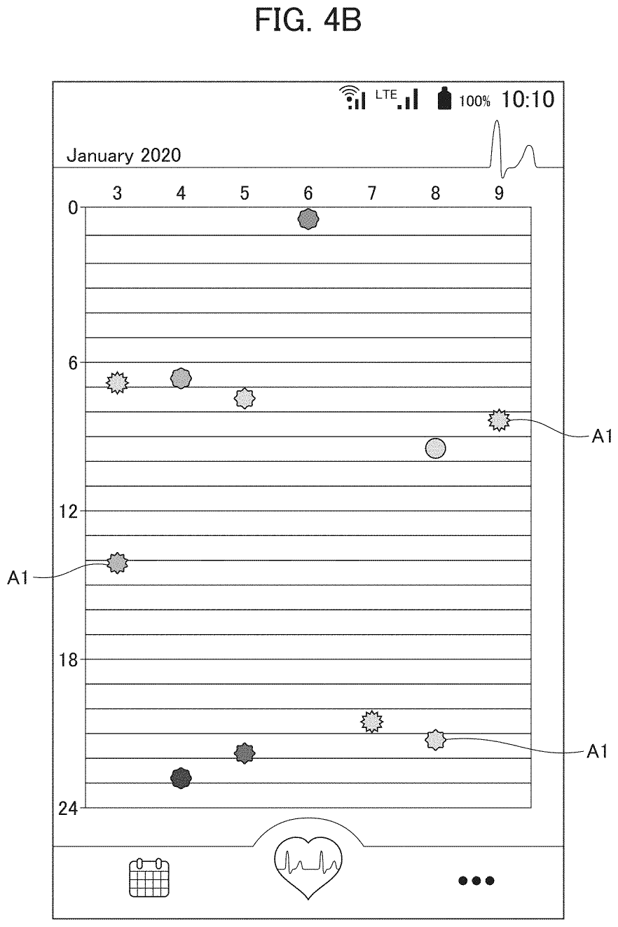

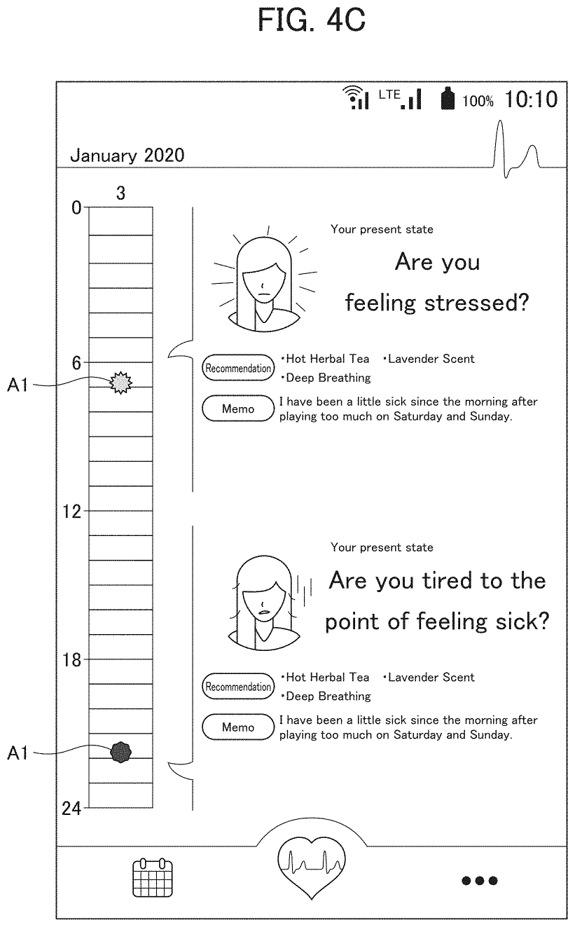

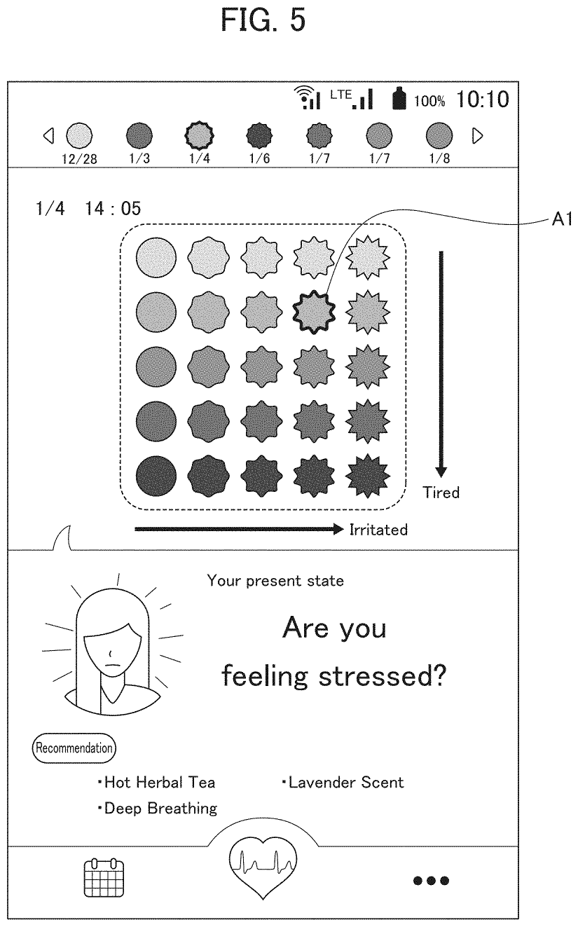

FIGS. 4A to 4C and FIG. 5 are diagrams each illustrating an example of what is displayed by the display-processing unit 28. As illustrated in FIG. 4A, the display-processing unit 28 may display the state-indicating graphics A1, A2 indicating the user's mental and physical state in chronological order in a monthly calendar format that shows the days of a month. As illustrated in FIG. 4B, the display-processing unit 28 can be configured to display the state-indicating graphics A1, A2 indicating the user's mental and physical state in chronological order in a weekly calendar format that shows the days of a week (e.g., along the horizontal axis) and the hours of each day (e.g., along the vertical axis). Alternatively, as illustrated in FIG. 4C, the display-processing unit 28 can be configured to display the state-indicating graphics A1, A2 indicating the mental and physical state in chronological order in a daily schedule format that shows the hours of a day. Alternatively, as illustrated in FIG. 5, the display-processing unit 28 can be configured to display a result of individual evaluation of each of the state-indicating graphics A1, A2 indicating the user's mental and physical state.

Each of the state-indicating graphics A1, A2 and criterial state-indicating graphics B1, B2 to be described later represents a level of the user's mental and physical state in a distinguishable manner by a change in the respective graphic. The mental and physical state may include at least two of a degree of stress, a degree of brain fatigue, and a degree of muscle fatigue. In this case, each of the state-indicating graphics A1, A2 and the criterial state-indicating graphics B1, B2 to be described later may be a single graphic that represents the at least two degrees included in the mental and physical state in a distinguishable manner by means of different display attributes. Each of the different display attributes is color, shape, size, or pattern. The display-processing unit 28 displays respective levels of the mental and physical state in a distinguishable manner by means of a change in the display attributes.

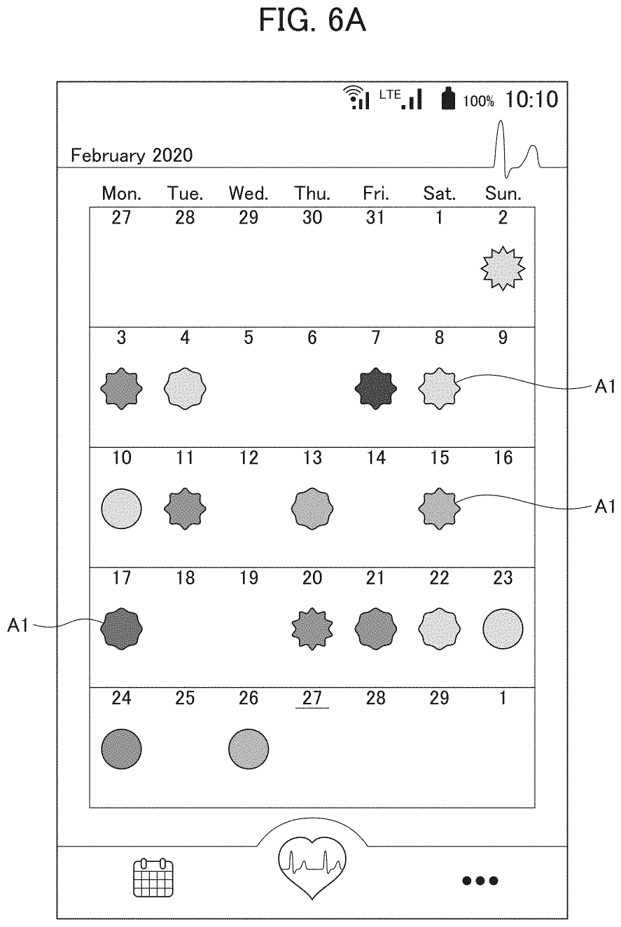

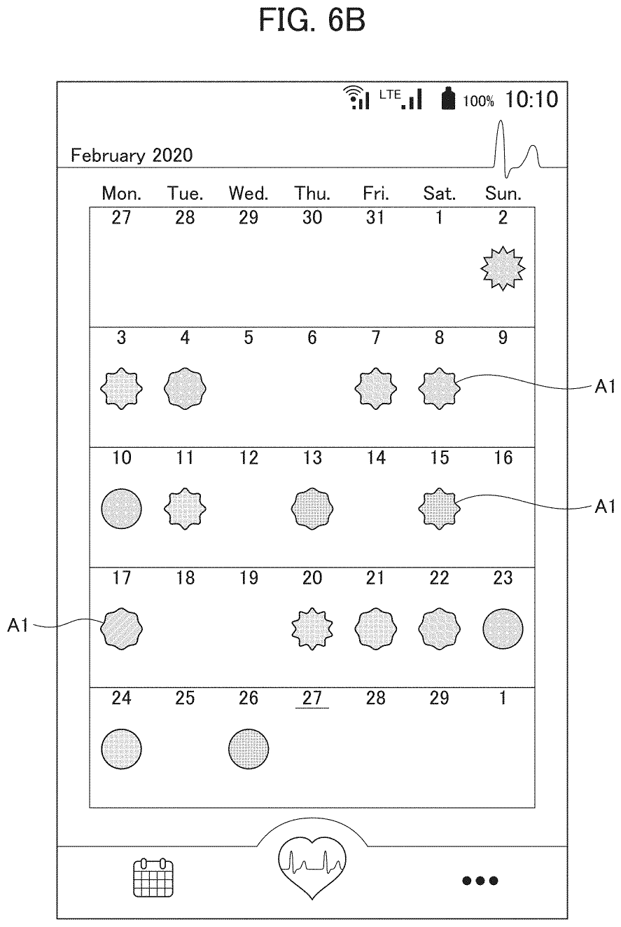

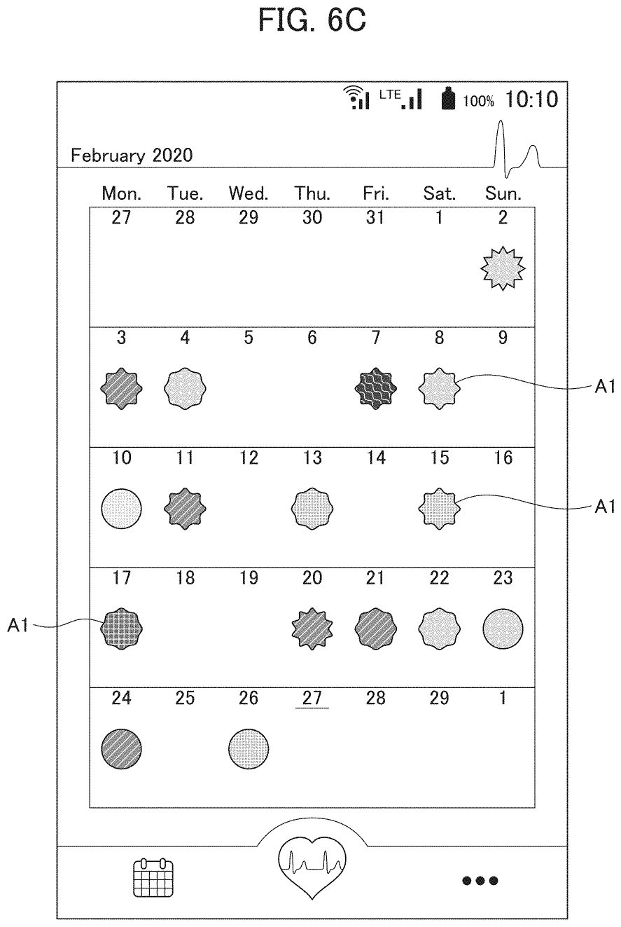

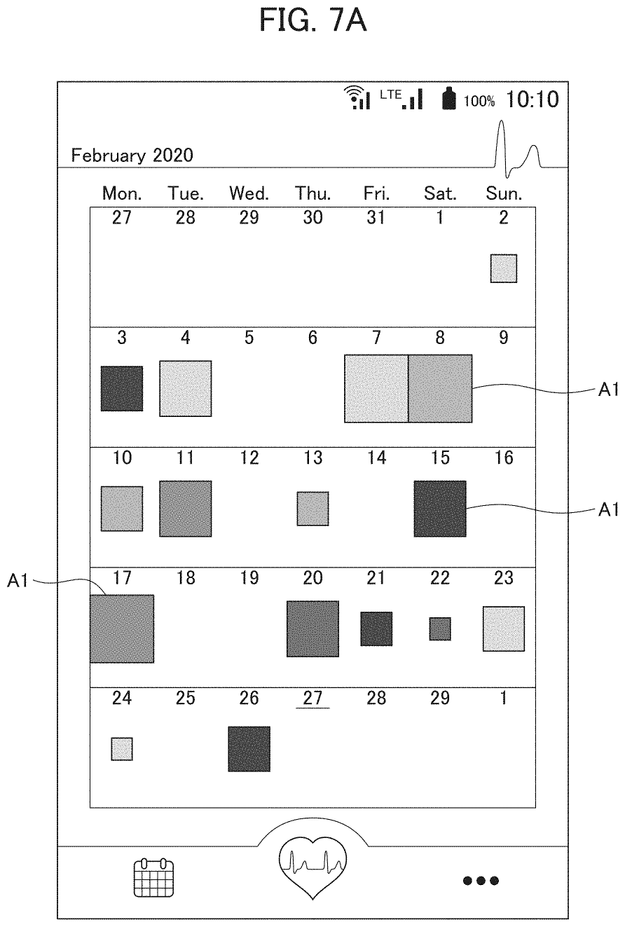

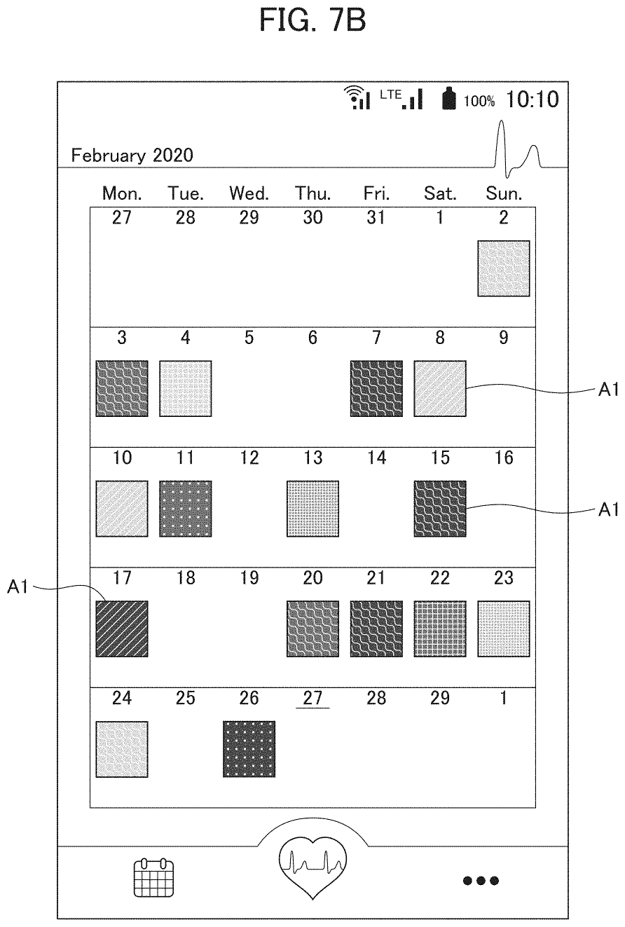

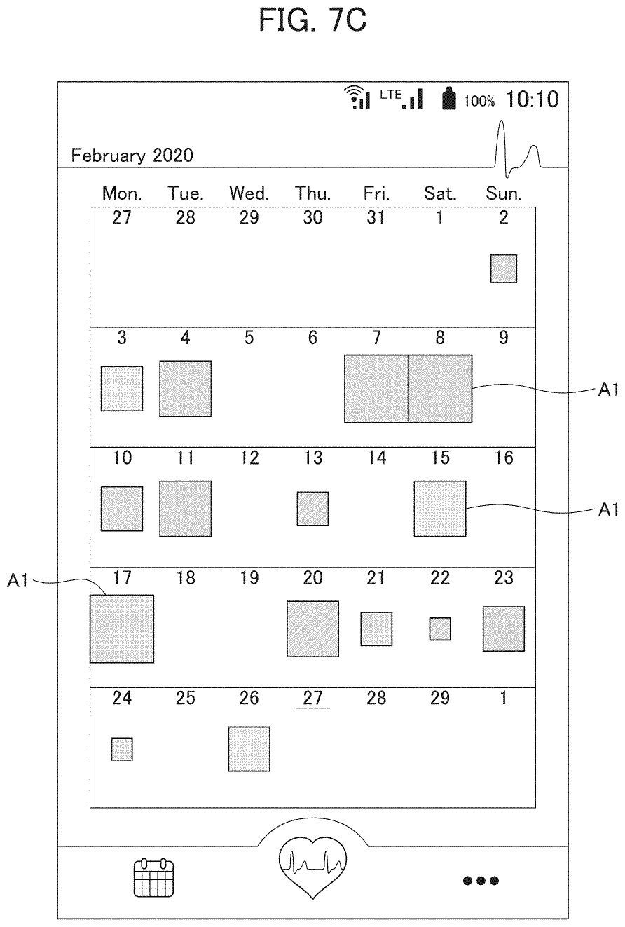

FIGS. 6A to 6C and FIGS. 7A to 7C are diagrams illustrating examples of the state-indicating graphics. In a case where the mental and physical state includes two types of degrees, namely, the degree of stress and the degree of brain fatigue, the degree of brain fatigue is represented by means of, for example, a change in the color as one display attribute of the state-indicating graphic A1, as illustrated in FIG. 6A. Color is determined depending on hue, lightness, and saturation. For example, the healthier the user is, the closer to one hue the color becomes, while the more tired the user is, the closer to the opposite hue the color becomes. Alternatively, the healthier the user is, the higher the brightness becomes, while the more tired the user is, the lower the brightness becomes. Alternatively, the healthier the user is, the more intensive the saturation becomes, while the more tired the user is, the less intensive the saturation becomes.

On the other hand, the degree of stress is represented by, for example, a change in the shape as one display attribute of the state-indicating graphic A1. For example, a zigzag of the circular circumference of the shape may be changed. For example, the zigzag becomes more round and decreases in number as the stress decreases, while the zigzag becomes sharper and increases in number as the stress increases.

Alternatively, as illustrated in FIG. 6B, the degree of brain fatigue may be represented by, for example, a change in the pattern as one display attribute of the state-indicating graphic A1. For example, the healthier the user is, the coarser the pattern becomes, while the more tired the user is, the denser the pattern becomes. On the other hand, the degree of stress is represented by, for example, a change in the shape as one display attribute of the state-indicating graphic A1, in the same manner as above.

In a case where the mental and physical state includes three types of degrees, namely, the degree of stress, the degree of brain fatigue, and the degree of muscle fatigue, as illustrated in FIG. 6C, the degree of brain fatigue is represented by, for example, a change in the color as one display attribute of the state-indicting graphic A1, in the same manner as above. On the other hand, the degree of muscle fatigue is represented by, for example, a change in the pattern as one display attribute of the state-indicating graphic A1, in the same manner as above. Further, the degree of stress is represented by, for example, a change in the shape as one display attribute of the state-indicating graphic A1, in the same manner as above.

Alternatively, in a case where the mental and physical state includes two types of degrees, namely, the degree of stress and the degree of brain fatigue, as illustrated in FIG. 7A, the degree of brain fatigue is represented by, for example, a change in the color as one display attribute of the state-indicting graphic A1, in the same manner as above. On the other hand, the degree of stress is represented by, for example, a change in the size as one display attribute of the state-indicating graphic A1. For example, the less the stress is, the smaller the size becomes, while the more the stress is, the larger the size becomes.

Alternatively, as illustrated in FIG. 7B, the degree of brain fatigue is represented by, for example, a change in the color as one display attribute of the state-indicating graphic A1, in the same manner as above. On the other hand, the degree of stress is represented by, for example, a change in the pattern as one display attribute of the state-indicating graphic A1.

Alternatively, as illustrated in FIG. 7C, the degree of brain fatigue is represented by means of, for example, a change in the pattern as one display attribute of the state-indicating graphic A1, in the same manner as above. On the other hand, the degree of stress is represented by, for example, a change in the size as one display attribute of the state-indicating graphic A1, in the same manner as above.

In the above-described way, one graphic represents two or more types of degrees included in the mental and physical state by the different display attributes. This feature allows the user to grasp the two or more degrees included in his/her mental and physical state simultaneously and intuitively.

Here, when a plurality of state-indicating graphics are displayed in parallel as in the case of the calendar format, it is expected to be difficult for the user to recognize a change in a certain display attribute, such as pattern or size, on a small display device (e.g., a smartphone). Further, the impression of a change in a certain display attribute, such as pattern or size, is expected to be less strong than the impression of a change in another display attribute, such as color or shape. Therefore, in a case where a change in the display attribute, such as pattern or size, is employed, it is expected that the user will not be able to correctly recognize his/her mental and physical state.

To address this issue, it is preferable to employ two display attributes, namely, color and shape, among the display attributes including color, shape, size, and pattern, as in the example illustrated in FIG. 6A. This feature allows the user to correctly recognize his/her mental and physical state.

Reference is made to FIG. 4A again. When the mental and physical state has a plurality of mental and physical states during one day, the display-processing unit 28 may display the state-indicating graphic A2 that indicates a representative one of the mental and physical states of the one day. In the example illustrated in FIG. 4A, the display-processing unit 28 may display the state-indicating graphic A2, indicating the representative mental and physical state of the day, in a larger size, and may display the other state-indicating graphics in a smaller size. Further, the display-processing unit 28 may display the state-indicating graphics together with the corresponding measurement time.

The representative mental and physical state of one day may be an average or a median of the plurality of mental and physical states. Alternatively, the representative mental and physical state of one day may be the first measured one or the last measured one among the plurality of mental and physical states, or may be one measured at intermediate time among the plurality of mental and physical states.

In the case where the mental and physical state has a plurality of mental and physical states during one day, if all the states are displayed as graphics of the same size, it is difficult for the user to understand a difference between the mental and physical states during the day, and to recognize fluctuation that has taken place during a month and fluctuation taking place during a week. To address this issue, the state-indicating graphic A2, which indicates the representative mental and physical state of one day, is displayed or displayed in a larger size. This feature makes it easier for the user to understand a difference between the mental and physical states during one day, and to recognize fluctuation that has taken place during a month and fluctuation that has taken place during a week.

As illustrated in FIG. 4A, the display-processing unit 28 displays, as the criterial state-indicating graphic B1, B2, at least one of the state-indicating graphics A1, A2 that corresponds to a criterion of the mental and physical state, the criterion being set on a user-to-user basis. The display-processing unit 28 displays the criterial state-indicating graphic B1, B2 such that it is distinguishable from the remainder of the other state-indicating graphics A1, A2. Thus, based on the criterial state-indicating graphic B1, B2, or for example, while comparing the state-indicating graphics A1, A2 with the criterial state-indicating graphic B1, B2, the user can intuitively grasp his/her mental and physical state substantially in agreement with his/her individual perception.

The criterion of the mental and physical state or the criterial state-indicating graphic B1, B2 can be manually set by the user. For example, the user can manually select and set, from the state-indicating graphics A1, A2 displayed on the display unit 25, one state-indicating graphic corresponding to the criterion of the mental and physical state, by operating the operation unit 24. The display-processing unit 28 can be configured to determine the one state-indicating graphic corresponding to the criterion of the mental and physical state, which has been manually set by the user, as the criterial state-indicating graphic B1, B2. Alternatively, the criterion of the mental and physical state or the criterial state-indicating graphic B1, B2 can be automatically set by the display-processing unit 28.

The display-processing unit 28 can be configured to set the criterion of the mental and physical state on a user-to-user basis, based on one or more of the following items: past mental and physical states; past measurement results of biometric information; life event information; basic information such as the age and gender of the user; a goal set by the user; a situation in which the mental and physical state is the best; and a situation in which the mental and physical state is the worst. The display-processing unit 28 determines the state-indicating graphic corresponding to the set criterion of the mental and physical state as the criterial state-indicating graphic B1, B2.

Setting the criterion of the mental and physical state based on two or more of the items allows the criterial state-indicating graphic B1, B2, which corresponds to the criterion of the mental and physical state to be displayed more appropriately.

Further, the display-processing unit 28 may adjust the entire levels of the mental and physical state according to the criterion of the mental and physical state set on a user-to-user basis, and may display the state-indicating graphics A1, A2 representing the adjusted levels of the mental and physical state. In other words, the display-processing unit 28 may shift and adjust the whole degrees of the display attributes of the state-indicating graphics A1, A2, in accordance with the adjusted levels of the mental and physical state.

As a result, not only the criterial state-indicating graphic B1, B2 representing the criterion of the mental and physical state, but also the whole state-indicating graphics A1, A2 indicating the mental and physical state are shifted and adjusted, so that the user can more intuitively grasp his/her mental and physical state substantially in agreement with his/her individual perception.

Further, the display-processing unit 28 can be configured to set a plurality of criteria of the mental and physical state on a user-to-user basis, and can also display a plurality of state-indicating graphics corresponding to the plurality of criteria of the mental and physical state as a plurality of criterial state-indicating graphics B1, B2. For example, as illustrated in FIG. 4A, the display-processing unit 28 sets, based on the past physical and mental states, two criteria of the physical and mental state, namely, a criterion of the situation in which the mental and physical state is the best and a criterion of the situation in which the mental and physical state is the worst, and then, displays two state-indicating graphics corresponding to the two criteria of the mental and physical state as the two criterial state-indicating graphics B1, B2. More specifically, the display-processing unit 28 displays the state-indicating graphic corresponding to the criterion of the situation of the best mental and physical state as the criterial state-indicating graphic B1, and the state-indicating graphic corresponding to the criterion of the situation of the worst mental and physical state as the criterial state-indicating graphic B2.

Displaying the plurality of criterial state-indicating graphics B1, B2 corresponding to the plurality of criteria of the mental and physical state enables the user to grasp his/her mental and physical state more intuitively, substantially in agreement with his/her individual perception. Especially, displaying the two criterial state-indicating graphics B1, B2, which correspond to the two criteria of the mental and physical state, i.e., the criterion of the situation of the best mental and physical state and the criterion of the situation the worst mental and physical state, enables the user to recognize how much his/her mental and physical state has fluctuated.

Here, a good physical and mental state refers to a situation that falls under one or more of the following: a situation where the user is aware that his/her mind and body are both healthy; a situation where the user is not experiencing fatigue, as a consequence of sufficient hours of sleep; a situation where the user feels refreshed in the morning, as a consequence of sufficient hours of sleep; and the best situation automatically selected based on measurement data of the user's past mental and physical states.

On the other hand, a poor mental and physical state refers to a situation that falls under one or more of the following: a situation where the user is aware that his/her mind and body are both unhealthy; a situation where the user is experiencing fatigue due to insufficient hours of sleep; a situation where the user feels fatigue or malaise due to an excessively long activity time; the worst situation selected automatically based on the measurement data of the user's past mental and physical states; and a situation where the user is aware that he/she is experiencing fatigue as, for example, in the definition by Japanese Society of Fatigue Science: “Fatigue is a state of decline of capacity for physical activity, accompanied with a peculiar discomfort and desire for rest, the state resulting from excessive physical and mental activity or illness”.

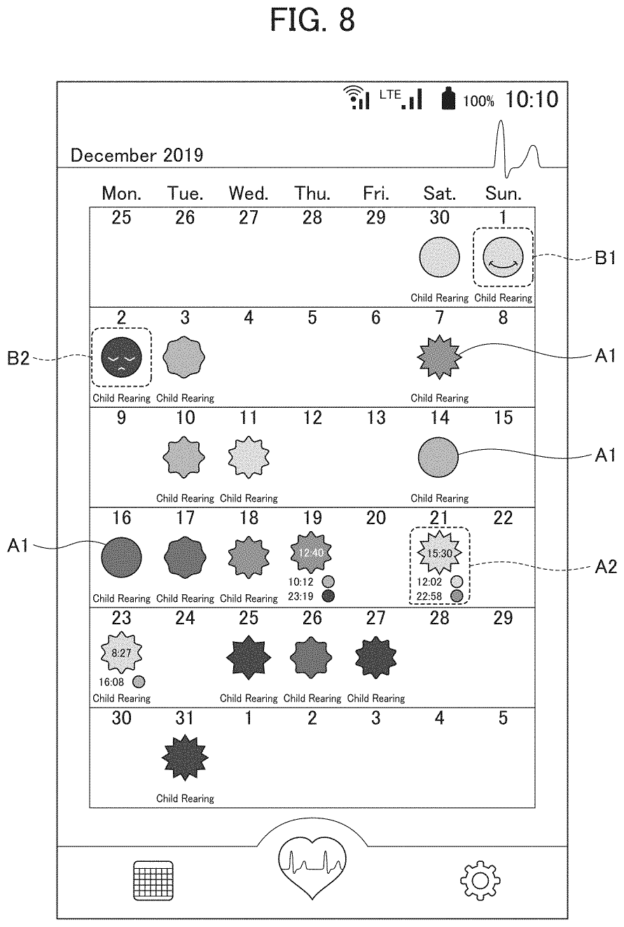

Alternatively, as illustrated in FIG. 8, for example, the display-processing unit 28 can be configured to adjust, based on the past mental and physical states and the life event information (e.g., pregnancy or child rearing), the whole levels of the past mental and physical states according to the life event information, and can also display the state-indicating graphics A1, A2 and the criterial state-indicating graphics B1, B2 so that all the graphics represent the respective adjusted levels of the mental and physical state. In other words, the display-processing unit 28 may shift and adjust the whole degrees of the display attributes of the state-indicating graphics A1, A2 and the criterial state-indicating graphics B1, B2, in accordance with the adjusted levels of the mental and physical state. Further, the display-processing unit 28 may display information indicating the life event information (e.g., “pregnant” or “child rearing”).

For example, during a certain life event, such as pregnancy or child rearing, the mental and physical state tends to deteriorate, in comparison with usual life events. In such a case, the whole mental and physical state and the criteria are shifted and adjusted, and information indicating the life event information, such as “pregnant” or “child rearing”, is displayed, so that the user can intuitively grasp his/her mental and physical state substantially in agreement with his/her individual perception, while recognizing the fluctuation of the mental and physical state due to the life event.

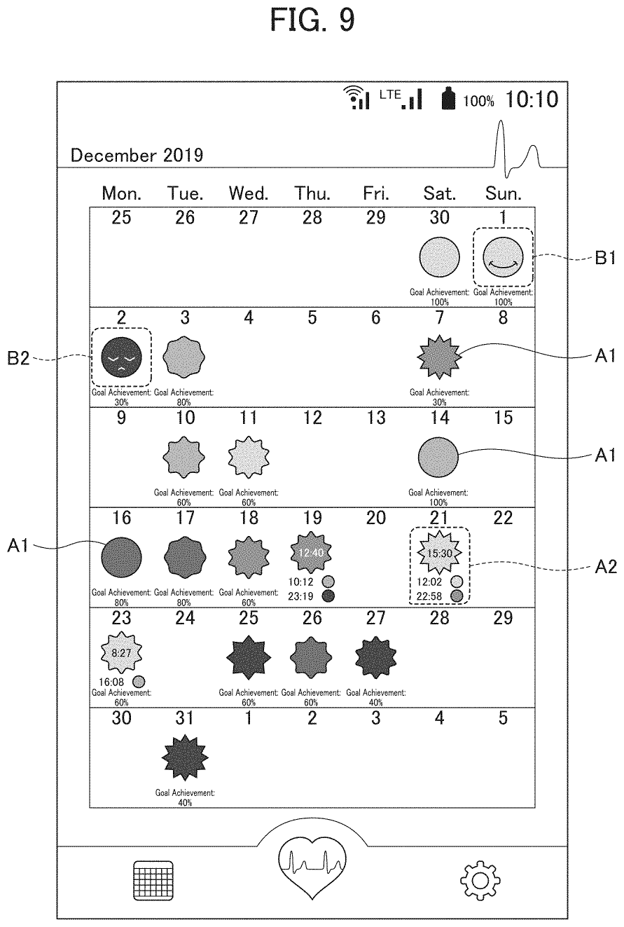

Alternatively, for example, as illustrated in FIG. 9, the display-processing unit 28 can be configured to set criteria of the mental and physical state on a user-to-user basis, based on the past mental and physical states and the goal, and may display criterial state-indicating graphics B1, B2 corresponding to the set criteria of the mental and physical state. Further, the display-processing unit 28 can be configured to display information indicating the goal (e.g., a goal achievement rate).

Displaying the information indicating the goal set by the user, such as the “goal achievement rate”, allows the user to intuitively grasp his/her mental and physical state substantially in agreement with his/her individual perception, while recognizing the goal. This feature makes it easy for the user to perform behavior modification and to improve performance.

Further, the display-processing unit 28 can be configured for updating the criterion/criteria of the mental and physical state. The physical and mental state fluctuates during a month or during a year depending on the user's lifestyle. The feature in which the criterion/criteria of the mental and physical state can be updated allows the user to correctly analyze his/her mental and physical state in detail on a time-by-time basis.

Further, in response to an operation of the operation unit 24 by the user, the display-processing unit 28 can perform switching between the following display formats: the monthly calendar format of FIG. 4A that shows the days of a month; the weekly calendar format of FIG. 4B that shows the days of a week; and the daily schedule format of FIG. 4C that shows the hours of a day. This feature allows the user to manage and analyze changes in his/her mental and physical state in various units such as month, week, and day. This feature makes it easy for the user to perform behavior modification.

Further, as illustrated in FIG. 5, the display-processing unit 28 can be configured to display a result of individual evaluation of the state-indicating graphics. For example, as shown in the upper half of FIG. 5, the horizontal axis represents the degree of stress while the vertical axis represents the degree of brain fatigue. This feature allows the user to manage and analyze changes in his/her mental and physical state.

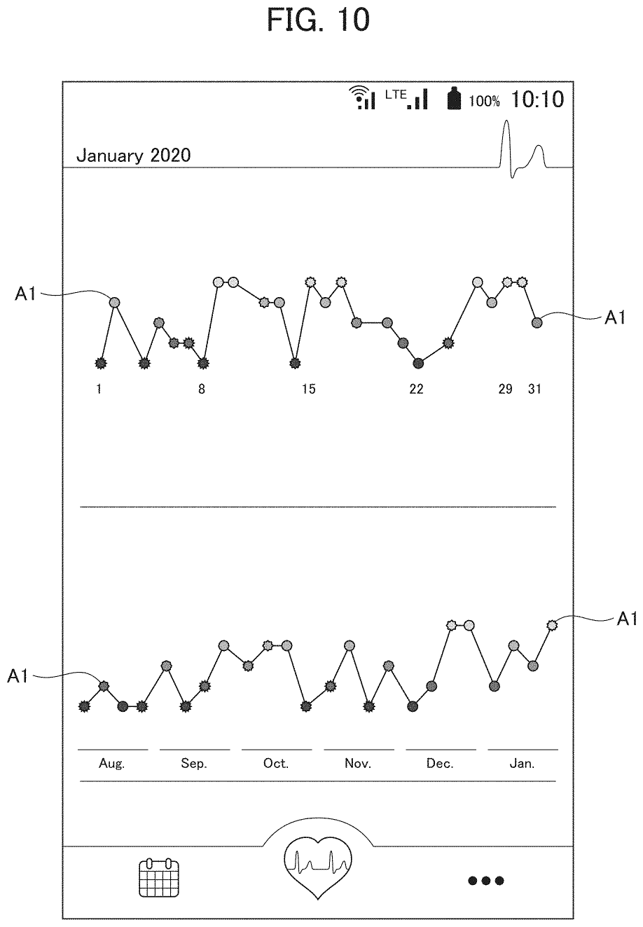

Further, in response to an operation of the operation unit 24 by the user, the display-processing unit 28 can switch the calendar or schedule format display illustrated in FIGS. 4A to 4C to trend format display illustrated in FIG. 10. As illustrated in the upper half of FIG. 10, in the trend format display, the vertical axis represents the levels of mental and physical state, while the horizontal axis represents the days of a month. This trend format display allows the user to manage and analyze changes in his/her mental and physical state. As illustrated in the lower half of FIG. 10, in the trend format display, the vertical axis represents the levels of the mental and physical state while the horizontal axis represents the weeks or months of a year. This trend format display allows the user to conduct longer-term management and analysis of changes in his/her mental and physical state, the longer-term management and analysis being impossible with the monthly calendar format display. This feature makes it easy for the user to perform behavior modification.

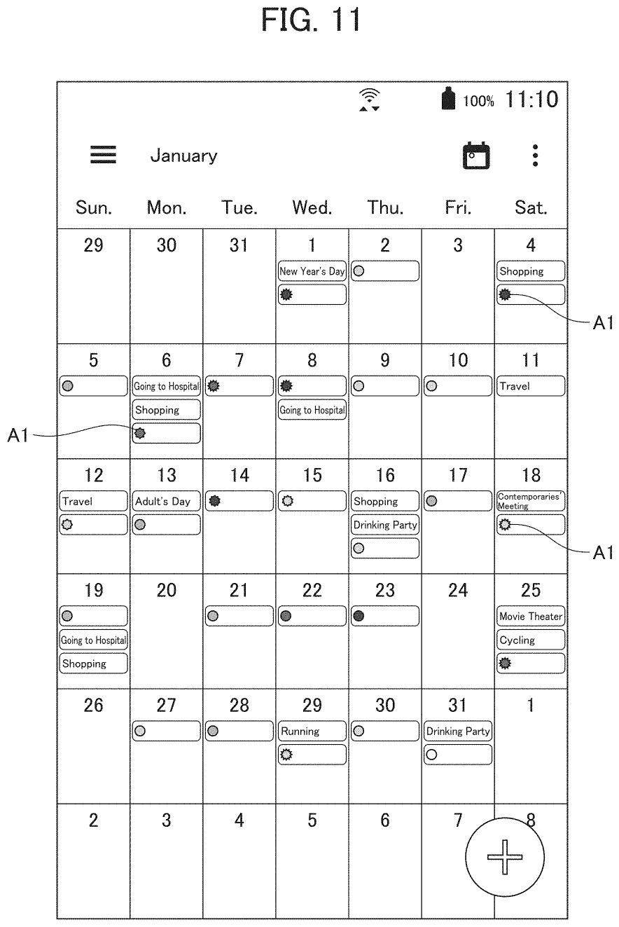

As illustrated in FIG. 11, the display-processing unit 28 can be linked with a schedule management function, and can display schedule information together with the state-indicating graphics A1, A2 and the criterial state-indicating graphics B1, B2 in the calendar format. This feature allows the user to manage and analyze a correlation between changes in his/her mental and physical state and the user's events. Thus, with this feature, the user can grasp what kind of action causes his/her mental and physical state to deteriorate and can easily perform behavior modification.

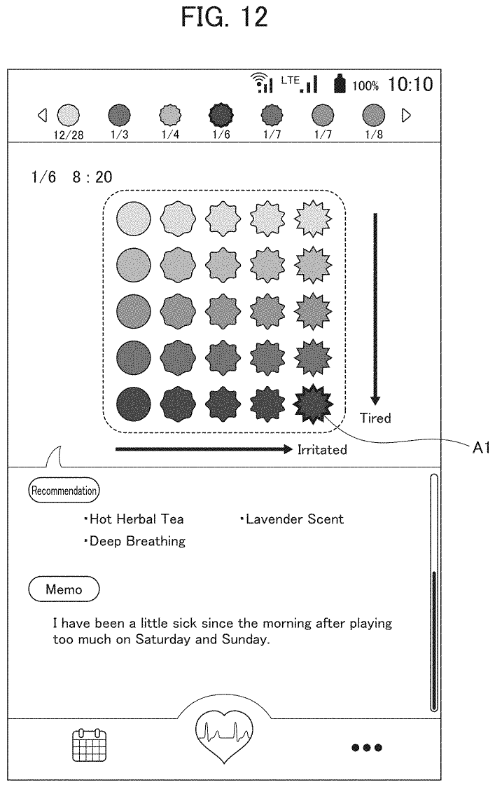

As illustrated in FIGS. 4C, 5, and 12, the display-processing unit 28 may display action information inputted by the user, together with the state-indicating graphics A1, A2 and the criterial state-indicating graphics B1, B2. For example, the user inputs, by operating the operation unit 24, a memo (e.g., action information) related to the user's action, with respect to the state-indicating graphic A1, A2 or the criterial state-indicating graphic B1, B2 displayed on the display unit 25. The storage unit 26 then stores the memo (e.g., action information) in association with the state-indicating graphic A1, A2 or the criterial state-indicating graphic B1, B2, or in association with the mental and physical state corresponding to the state-indicating graphic A1, A2 or the criterial state-indicating graphic B1, B2. The display-processing unit 28 displays the memo (e.g., action information), together with the state-indicating graphic A1, A2 or the criterial state-indicating graphic B1, B2.

This feature allows the user to manage and analyze a correlation between changes in his/her mental and physical state and the memo (e.g., action information). Thus, with this feature, the user can grasp what kind of action causes his/her mental and physical state to deteriorate and can easily perform behavior modification.

Further, as illustrated in FIGS. 4C, 5, and 12, the display-processing unit 28 can be configured to display inquiry information that says “Are you feeling stressed?”, according to the levels of the mental and physical state. This feature makes it possible to provide an example of evaluation of the level of the mental and physical state, thereby allowing the user to easily perform behavior modification. Further, the display-processing unit 28 may display an example of easy behavior modification in the form of a “recommendation”. This feature makes it easy for the user to perform behavior modification.

In accordance with an exemplary aspect, the display-processing unit 28 includes an arithmetic processor, such as a central processing unit (CPU), a digital signal processor (DSP), or a field-programmable gate array (FPGA). Various functions of the display-processing unit 28 are performed by way of execution of, for example, programs (e.g., applications) stored in the storage unit 26. The programs (e.g., applications) may be provided through the network, or may be recorded on and provided from a computer readable storage medium 40, such as a compact disc read only memory (CD-ROM) or a digital versatile disc (DVD). Examples of the storage medium include non-transitory tangible media.

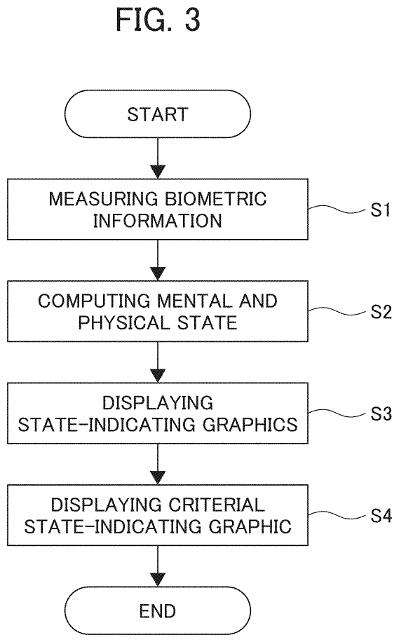

Next, with reference to FIG. 3, a mental and physical state-displaying operation performed by the above-described mental and physical state display system 1 and the above-described mental and physical state display device 20 will be described. FIG. 3 is a flowchart of the mental and physical state-displaying operation performed by the mental and physical state display system 1 and device 20 according to the present embodiment.

First, the biometric sensor 10 measures biometric information of the user under the control of the display device 20 (S1). The sensor 10 transmits the measured biometric information to the display device 20. The display device 20 transmits the result of measurement of the biometric information to the host device 30 through the network 5.

Next, the mental and physical state computation device 35 computes a mental and physical state of the user based on the result of measurement of the biometric information (S2). The host device 30 transmits the computed mental and physical state to the display device 20.

Next, the display device 20 chronologically displays state-indicating graphics A1, A2 that indicate the calculated mental and physical state and that represent levels of the mental and physical state in a distinguishable manner by a change in the respective graphics, in a monthly calendar format, a weekly calendar format, or a daily schedule format (S3).

Next, the display device 20 displays at least one of the state-indicating graphics A1, A2 that corresponds to a criterion of the mental and physical state as the criterial state-indicating graphic B1, B2, the criterion being set on a user-to-user basis, in such a manner that the criterial state-indicating graphic B1, B2 is distinguishable from the remainder of the state-indicating graphics A1, A2 (S4).

As described above, the mental and physical state display device 20 of the present embodiment, which displays the user's mental and physical state in the form of the state-indicating graphics A1, A2 in the calendar or schedule format, allows the user to intuitively grasp fluctuation of his/her mental and physical state that has taken place during a month or a week due to the user's lifestyle and biorhythm. This feature enables the user to perform behavior modification and to improve performance.

Further, the mental and physical state display device 20 of the present embodiment displays the criterial state-indicating graphic B1, B2 corresponding to the criterion of the mental and physical state set on a user-to-user basis. Thus, based on the criterial state-indicating graphic B1, B2, and for example, while comparing the state-indicating graphics A1, A2 with the criterial state-indicating graphic B1, B2, the user can intuitively grasp his/her mental and physical state substantially in agreement with his/her individual perception.

In the foregoing, one embodiment of the present disclosure has been described. It is noted that the above-described embodiment is not intended to limit the present disclosure, and various variations, modification, and combinations thereof can be made to the present disclosure. For example, in the above-described embodiment, the mental and physical state display device 20, which displays two or more types of degrees (e.g., a degree of stress, a degree of brain fatigue, a degree of muscle fatigue) included in the mental and physical state in the form of a single state-indicating graphic, has been descried as an example. However, this is a non-limiting example. The present disclosure is applicable to a mental and physical state display device that displays at least one degree (e.g., a degree of stress, a degree of brain fatigue, a degree of muscle fatigue) included in the mental and physical state in the form of a single state-indicating graphic.

### EXPLANATION OF REFERENCE NUMERALS

1: Mental and Physical State Display System
5: Network
10: Biometric Sensor
20: Mental and Physical State Display Device
21: Communication Unit
22: Communication Unit
24: Operation Unit
25: Display Unit
26: Storage Unit
28: Display-Processing Unit
30: Host Device
35: Mental and Physical State Computation Device
40: Storage Medium
Ap: Access Point
A1, A2: State-Indicating Graphic
B1, B2: Criterial State-Indicating Graphic

What is claimed is:

1. A mental and physical state display device comprising:
a display unit; and
a display-processing unit configured to display, on the display unit, state-indicating graphics in chronological order in at least one of a monthly calendar format, a weekly calendar format, and a daily schedule format, with the state-indicating graphics indicating a mental and physical state determined based on a result of a measurement of biometric information of a user, and representing levels of the mental and physical state in a distinguishable manner by a change in the respective state-indicating graphic,
wherein the display-processing unit is configured to display at least one of the state-indicating graphics that corresponds to a criterion of the mental and physical state as a criterial state-indicating graphic that is distinguishable from a remainder of the state-indicating graphics, with the criterion being set on a user-to-user basis.

2. The mental and physical state display device according to claim 1,
wherein the mental and physical state includes at least one of a degree of stress and a degree of fatigue, and
wherein the degree of fatigue includes a degree of brain fatigue and a degree of muscle fatigue.

3. The mental and physical state display device according to claim 1, wherein the display-processing unit is configured to switch display between the monthly calendar format, the weekly calendar format, and the daily schedule format.

4. The mental and physical state display device according to claim 1, wherein the display-processing unit is configured to switch display from the respective calendar formats and the schedule format to a trend format.

5. The mental and physical state display device according to claim 1, further comprising:
a storage unit configured to store schedule information of the user,
wherein the display-processing unit is configured to display the schedule information together with the state-indicating graphics and the criterial state-indicating graphic.

6. The mental and physical state display device according to claim 1, further comprising:
an operation unit configured to receive an input for action information related to an action of the user with respect to the state-indicating graphics or the criterial state-indicating graphic displayed on the display unit; and
a storage unit configured to store the action information in association with the state-indicating graphics or the criterial state-indicating graphic, or in association with the mental and physical state corresponding to the state-indicating graphics or the criterial state-indicating graphic,
wherein the display-processing unit is configured to display the action information together with the state-indicating graphics and the criterial state-indicating graphic.

7. The mental and physical state display device according to claim 2,
wherein the mental and physical state includes at least two of the degree of stress and the degree of fatigue,
wherein each of the state-indicating graphics and the criterial state-indicating graphic is a single graphic that represents the at least two degrees included in the mental and physical state in a distinguishable manner by different display attributes,
wherein each of the different display attributes is at least one of color, shape, size, and pattern, and
wherein the display-processing unit is configured to display each of the levels of the mental and physical state in a distinguishable manner by a change in each display attribute.

8. The mental and physical state display device according to claim 2,
wherein the mental and physical state includes two of the degree of stress and the degree of fatigue,
wherein each of the state-indicating graphics and the criterial state-indicating graphic is a single graphic that represents the two degrees included in the mental and physical state in a distinguishable manner by two different display attributes,
wherein the two display attributes are color and shape, respectively, and
wherein the display-processing unit is configured to display each of the levels of the mental and physical state in a distinguishable manner by a change in each display attribute.

9. The mental and physical state display device according to claim 1, wherein the display-processing unit is further configured to:
set the criterion of the mental and physical state on the user-to-user basis, based on one or more selected from past mental and physical states, measurement results of past biometric information, life event information, age, gender, a goal, an best situation for the mental and physical state, and a worst situation for the mental and physical state, and
determine one of the state-indicating graphics that corresponds to the set criterion of the mental and physical state as the criterial state-indicating graphic.

10. The mental and physical state display device according to claim 9, wherein the display-processing unit is further configured to:
adjust whole levels of the mental and physical state according to the criterion of the mental and physical state set on the user-to-user basis, and
display the state-indicating graphics representing the adjusted levels of the mental and physical state.

11. The mental and physical state display device according to claim 9, wherein the display-processing unit is further configured to:
set, on the user-to-user basis, the criterion comprising a plurality of criteria of the mental and physical state, and
display the state-indicating graphics corresponding to the plurality of criteria of the mental and physical state, as the criterial state-indicating graphic comprising a plurality of criterial state-indicating graphics.

12. The mental and physical state display device according to claim 11, wherein the display-processing unit is further configured to:
set, based on the past mental and physical states, the criterion comprising two criteria of the mental and physical state that includes a criterion of the best situation for the mental and physical state, and a criterion of the worst situation for the mental and physical state is the worst, and
display two of the state-indicating graphics corresponding to the two criteria of the mental and physical state, as the criterial state-indicating graphic comprising two criterial state-indicating graphics.

13. The mental and physical state display device according to claim 9, wherein the display-processing unit is further configured to:
adjust, based on the past mental and physical states and the life event information, whole levels of the past mental and physical states according to the life event information, and
display the state-indicating graphics and the criterial state-indicating graphic that represent the adjusted levels of the mental and physical state, together with information indicating the life event information.

14. The mental and physical state display device according to claim 9, wherein the display-processing unit is further configured to:
set, on the user-to-user basis, the criterion of the mental and physical state based on the past mental and physical states and the goal, and
display the criterial state-indicating graphic corresponding to the set criterion of the mental and physical state, together with information indicating the goal.

15. The mental and physical state display device according to claim 1, wherein the display-processing unit is configured to update the criterion of the mental and physical state.

16. The mental and physical state display device according to claim 1, wherein, when the mental and physical state has a plurality of mental and physical states during one day, the display-processing unit is configured to display the state-indicating graphic that indicates a representative state of the plurality of mental and physical states of the day.

17. A mental and physical state display system comprising:
a sensor configured to measure biometric information of a user;
a computation device configured to determine a mental and physical state of the user based on a measurement result of the biometric information;
the mental and physical state display device according to claim 1 that displays, in the calendar or schedule format, the state-indicating graphics indicating the computed mental and physical state and the criterial state-indicating graphic corresponding to the criterion of the mental and physical state set on the user-to-user basis; and
a host device configured to communicate with the mental and physical state display device.

18. A mental and physical state display method comprising:
displaying state-indicating graphics in chronological order in at least one of a monthly calendar format, a weekly calendar format, and a daily schedule format, with the state-indicating graphics indicating a mental and physical state computed based on a result of a measurement of biometric information of a user, and representing levels of the mental and physical state in a distinguishable manner by a change in the respective state-indicating graphic; and
displaying at least one of the state-indicating graphics that corresponds to a criterion of the mental and physical state as a criterial state-indicating graphic that is distinguishable from a remainder of the state-indicating graphics, with the criterion being set on a user-to-user basis.

19. A computer-readable storage medium constructed to store a program, which, when executed by a processor of a computer, causes the computer to:
display state-indicating graphics in chronological order in at least one of a monthly calendar format, a weekly calendar format, and a daily schedule format, with the state-indicating graphics indicating a mental and physical state computed based on a result of a measurement of biometric information of a user, and representing levels of the mental and physical state in a distinguishable manner by a change in the respective state-indicating graphic; and
display at least one of the state-indicating graphics that corresponds to a criterion of the mental and physical state as a criterial state-indicating graphic that is distinguishable from a remainder of the state-indicating graphics, with the criterion being set on a user-to-user basis.

20. The computer-readable storage medium according to claim 19, wherein the mental and physical state includes at least one of a degree of stress and a degree of fatigue, with the degree of fatigue including a degree of brain fatigue and a degree of muscle fatigue.
